(19) United States
(12) Patent Application Publication
(10) Pub. No.: US 2008/0220104 A1
(43) Pub. Date: Sep. 11, 2008

(54) Compositions for producing satiety

(76) Inventor: John V. Cappello, Myrtle Beach, SC (US)

Correspondence Address: Lawrence J. Shurupoff, 16651 Topanga Lane, Delray Beach, FL, 33484, US

(21) Appl. No.: 11/716,339

(22) Filed: Mar. 8, 2007

(51) Int. Cl. A61K 31/405; A61K 31/19; A61K 31/44; A61K 31/70; A61K 36/48; A61K 36/81; A61P 43/00

(52) U.S. Cl. 424/757; 424/773; 514/159; 514/23; 514/345; 514/355; 514/356; 514/419

(57) ABSTRACT

Long term weight loss can be achieved and maintained with a formulation of over-the-counter life-enhancing dietary supplements. One or more supplements stimulate the release of serotonin within the brain and one or more supplements stimulate the release of cholecystokinin into the bloodstream. The combined potentiating effect of these two mechanisms on satiety enables greater weight loss than that previously achieved by the individual mechanisms acting alone.

### BACKGROUND AND SUMMARY

Being overweight or obese puts one at serious risk for developing many obesity-related illnesses including heart attack, stroke, type II diabetes and hypertension. Gall bladder disease, breast cancer, prostate cancer, colon cancer, sleep apnea, osteoarthritis and respiratory problems also pose a greater risk for those who are obese.

There are numerous products available on the market, both over-the-counter (OTC) and by prescription for controlling appetite and reducing body weight. A review of OTC products finds that many are based upon a predominance of empirical information. These OTC products typically contain caffeine, herbs of varying source and purity, and so-called fat blockers and carbohydrate blockers.

Weight control prescription formulations such as the amphetamine class can be dangerous and addictive. Newer advances of the serotonin reuptake variety and scientifically developed fat blockers are expensive and typically available only by prescription.

As detailed below, new compositions and formulations have been developed to provide a pair of weight loss mechanisms using proven weight loss technologies. This dual action approach to weight loss produces results beyond what an arithmetic summation of the individual technologies would produce. Little has been found in the literature about combining known mechanisms of weight loss, and nothing has been found using the formulations set forth below.

### DETAILED DESCRIPTION OF THE EMBODIMENTS

The combined use of L-tryptophan (tryptophan) such as in the form of 5-hydroxytryptamine (5-HT) to enhance serotonin production in humans, and dietary supplements which stimulate cholecystokinin (CCK) production in humans, along with additional optional ingredients has produced pronounced weight loss by creating a feeling of calmness, satisfaction, fullness and satiety for extended periods of time.

Prior uses of tryptophan are disclosed in U.S. Pat. Nos. 4,639,465 and 4,650,789, which are incorporated herein by reference in their entireties. Using the knowledge found in U.S. Pat. Nos. 4,639,465 and 4,650,789, which use tryptophan/serotonin science, a morbidly obese subject was placed on a formulation containing 100 mg of 5-HT, 100 mg of fructose, 20 mg of niacin, and 20 mg of vitamin B-6. The subject took one tablet 3 times a day, one half hour before meals. The subject's weight loss after 4 weeks was 3 pounds. This is in line with optimal information in the literature and also in line with expectations from the prescription medications which utilize the drug Sibutramine to affect serotonin and norepinephrine.

It is also reported in the literature that long chain fatty acids, both saturated and unsaturated, are capable of stimulating the release of cholecystokinin, a factor secreted by the gall bladder which promotes the feeling of satiety. L-phenylalanine (phenylalanine) is also known to do the same. While there are other substances which can stimulate cholecystokinin and are considered within the scope of this invention, it is believed that long chain fatty acids and L-phenylalanine are particularly beneficial

The same morbidly obese subject was placed on a formulation provided in a capsule containing 1200 mg. of saturated and unsaturated fat derived from fish oil and borage oil. Once again, instructions were given to the subject to take the capsule three times per day, one half hour before meals. Weight loss was recorded after 4 weeks and totaled 3 pounds. This too is in keeping with what has been reported as possible weight loss, though not always a guarantee of such weight loss.

In order to test the theory that combining these two mechanisms and technologies is superior to the arithmetic or aggregate effect of each, the same morbidly obese subject was told to take both formulations from the prior two tests at the same time, one half hour before meals. As with the other examples, no specific diet instructions were given. Weight loss after 4 weeks in this instance totaled 18 pounds, far beyond the maximum expected by arithmetic summation of the prior two tests.

It is believed that such results beyond arithmetic aggregation can be attained in morbidly obese subjects and to some extent in other overweight individuals for several reasons. In general, weight loss subjects are known to develop tolerance to various treatments relying on a single weight loss biochemical mechanism. Because the combination of ingredients offers multiple potentiating mechanisms which produce serotonin and CCK, it may be more difficult for the individual to develop tolerance over the short or intermediate term to more than one weight loss mechanism. The individual's body may take longer to react and develop tolerance to multiple weight loss mechanisms than to a single weight loss mechanism. Likewise, these two mechanisms appear to address many eating triggers including low serotonin, depression, carbohydrate craving, obsessive disorders, anxiety and pain.

In addition, serotonin signals to the brain resulting in a mental sense of satiety and wellness as well as a palpable sense of satiety of fullness in the stomach from CCK are reported from subjects taking a combination of tryptophan and fatty acids and/or phenylalanine.

The serotonin increasing formulation as outlined above, coupled with CCK stimulating formulations containing 400 mg to 2000 mg of saturated or unsaturated fatty acid or 100 mg to 500 mg of L-phenylalinine are effective for producing weight loss in humans. This is not to exclude other dietary supplements which promote these mechanisms and which are well known to those familiar with the art.

It is also possible to include other methods of weight loss science to further expand and improve weight loss results. One example is the addition of a proteinase inhibitor such as found in potatoes, soy and beans to the dual 5-HT and fatty acid/phenylalanine formulation described herein. This can increase the duration of satiety by prolonging the presence of CCK in the blood.

It is important to note that obesity as a disease is a chronic problem. The weight loss substances discussed herein are generally regarded as safe by the FDA and appear to have long term utility unlike many drugs which can produce toxic side effects after prolonged use. Moreover, the use of a number of different weight loss ingredients operating on different biochemical mechanisms appears to be resistant to tolerance build-up and therefore ideal for long term use.

The use of any form of tryptophan, such as 5-HT, in combination with one or more ingredients such as a fatty acid and/or L-phenylalinine provides the basis for effective long term weight loss. The additional ingredients noted above further improve the weight loss and satiety experienced by weight loss test subjects.

Initial results suggest that the longer the combination of 5-HT and long chain fatty acids are taken, the greater is the tendency for longer lasting endogenous regulation of appetite and maintenance of weight loss after a subject ceases taking the combination.

### EXAMPLE

Forty-eight subjects with body mass indexes (BMIs) of 30 or higher were identified. A BMI of 30 or more is categorized as obese.

These subjects were evenly divided into 3 groups of sixteen. Each group was given the same instructions regarding exercise (daily walks of about 30 minutes) and diet (a well balanced 1200 calorie diet) to limit variations in exercise and diet between groups.

The first group, group A, was given a 100 mg tablet of 5-hydroxytryptamine (5-HT) available over-the counter under the brand “Natrol”. This particular form of 5-HT is derived from griffonia seed and comes combined with the inert ingredients of rice flour, gelatin, silicon dioxide, water and magnesium stearate. However, any brand of OTC 5-HT can be used to increase serotonin. Subjects were told to take this tablet with a full glass of water one half hour before meals.

The second group, group B, was given a softgel capsule of fatty acids available over-the-counter under the brand “Multi-Oil” and available through GNC. Each capsule contains 15 calories, with 15 calories from fat. The total fat content is 1.5 grams, with zero grams of saturated fat, 0.5 grams of polyunsaturated fat and 0.5 grams of monosaturated fat. The fat is provided as oleic acid (omega 9) at 270 mg; EPA-omega-3 (eicosapentaenoic acid) at 180 mg; DHA-omega-3 (docosahexanoic acid) at 120 mg; linoleic acid (omega-6) at 217 mg, alpha linolenic acid (omega-3) at 71 mg; and GLA-omega-6 (gamma linolenic acid at 43 mg. Other labeled secondary ingredients are identified as fish body oil, gelatin, glycerin, flax seed oil, wheat germ oil, evening primrose oil, borage oil, black currant oil, natural lemon flavor, caramel color, vitamin E, and rosemary leaf extract. The composition also contains fish, soy beans and wheat. The purpose of providing fatty acids, such as long chain fatty acids is to increase cholecystokonin secretion and release CCK into the bloodstream. Each capsule was taken with a full glass of water one half hour before meals.

The third group, group C, was given the tablet of group A and the capsule of group B to increase both serotonin in the brain and cholecystokonin in the gut and bloodstream. Both the tablet and capsule were taken with a full glass of water one half hour before meals.

After 4 weeks, group A weight loss average was recorded at 3.5 pounds; group B weight loss average was recorded at 4.2 pounds, and group C weight loss average was recorded at 11.6 pounds.

It is believed that the weight loss attainable with morbidly obese subjects using the dual acting natural mechanisms of increasing serotonin and cholecystokinin was greater than the mathematical or arithmetic expectation of the summation of weight loss attributable to each individual mechanism. All of the formulations were well tolerated. A calming effect was reported by those in group A and group C.

In a post study observation, half of those subjects in group C reported no weight gain after 30 days of ceasing to use the dual acting formulation. It is believed that a longer term appetite regulation may be triggered over a long period of time after the formulation is discontinued as compared to other weight control formulations. It is also believed that the longer a subject takes the weight control formulations described herein using tryptophan and fatty acids, the longer the subject can maintain weight loss after discontinuing use of the tryptophan and fatty acids.

The appetite controlling effects of 5-HT can be enhanced by adding to the 5-HT and fatty acid/phenylalanine formulations one or more of a sugar such as fructose, niacin, nicotinamide, pyridosine (vitamin B6) and a salicylate such as acetylsalicylic acid (aspirin). Fructose can be added at a dosage of 250 mg or more, niacin and/or nicotinamide at a dosage of 25 mg or more, and aspirin at a dosage of 100 mg or more.

The appetite controlling effects of fatty acids and L-phenylalanine can be enhanced by adding to the 5-HT and fatty acid/phenylalanine formulations a proteinase inhibitor such as potatoes, soy and/or beans in any suitable form such as powders and liquids.

A particularly convenient form of administering a combined dosage of 5HTP and a fatty acid is through dual encapsulation wherein 5-HTP in a solid or powder form, and fatty acid in liquid of syrup form are provided in a single capsule. Each ingredient is individually encapsulated and these capsules are further encapsulated or combined as an integral or unitary assembly which can be ingested as a single unitary dose.

This facilitates ingestion of the formulations, as they may be taken with water in a single swallow. It also ensures that both ingredients are taken, as subjects can forget to take one or the other ingredients each time before meals. This further eliminates the need for multiple pill containers.

There has been disclosed heretofore the best embodiment of the invention presently contemplated. Obviously, numerous modifications and variations of the present invention are possible in light of the above teachings. It is therefore to be understood that within the scope of the appended claims, the invention may be practiced otherwise than as specifically described herein.

What is claimed is:

1. A dietary formulation, comprising:
tryptophan in an amount sufficient to produce a first weight loss in a human;
a fatty acid in an amount sufficient to produce a second weight loss in a human; and
said amounts of tryptophan and said fatty acid in combination producing a greater weight loss than that produced by said first and second weight losses.

2. The formulation of claim 1, wherein said tryptophan comprises 5-hydroxytryptophan.

3. The formulation of claim 1, wherein said fatty acid comprises a long chain fatty acid.

4. The formulation of claim 3, wherein said fatty acid comprises a saturated fatty acid.

5. The formulation of claim 3, wherein said fatty acid comprises an unsaturated fatty acid.

6. The formulation of claim 1, further comprising at least one ingredient selected from the group consisting of a sugar, niacin, nicotinamide, pyridoxine, a salicylate, and a proteinase inhibitor.

7. The formulation of claim 6, wherein said sugar comprises fructose, said salicyalte comprises acetylsalicylic acid, and said proteinase inhibitor comprises potato.

8. A dietary formulation, comprising:
a first ingredient for stimulating production of serotonin in an amount sufficient to produce a first weight loss in a human;
a second ingredient for stimulating production of cholecystokinin in an amount sufficient to produce a second weight loss in a human; and
wherein said amounts of said first and second ingredients in combination produce a third weight loss greater than said first and second weight losses.

9. The formulation of claim 8, wherein said first ingredient comprises tryptophan and said second ingredient comprises a fatty acid.

10. The formulation of claim 8, wherein said first ingredient comprises tryptophan and said second ingredient comprises phenylalanine.

11. The formulation of claim 8, wherein said first ingredient comprises 5-hydroxytryptophan and said second ingredient comprises a long chain fatty acid.

12. The formulation of claim 8, further comprising a proteinase inhibitor.

13. The formulation of claim 12, wherein said proteinase inhibitor is selected from the group consisting of potatoes, soy and beans.

14. The formulation of claim 8, wherein said second ingredient comprises at least one fatty acid selected from the group consisting of EPA-omega-3, DHA-omega-3, GLA-omega-3, linoleic acid, and alpha linolenic acid.

15. The formulation of claim 8, further comprising a third ingredient to further stimulate production of serotonin.

16. The formulation of claims 15, wherein said third ingredient is selected from the group consisting of a sugar, niacin, nicotinamide, pyridoxine and salicylate.

17. The formulation of claim 8, wherein said first ingredient comprises at least 100 mg of 5-hydroxytryptophan and said second ingredient comprises at least 400 mg of a long chain fatty acid.

18. A method of producing a feeling of satiety in a human subject, comprising:
orally ingesting a first ingredient which stimulates a release of serotonin comprising a first amount of tryptophan sufficient to independently produce a feeling of satiety in the subject; and
orally ingesting a second ingredient which stimulates release of cholecystokinin in an amount sufficient to independently produce a feeling of satiety in the subject.

19. The method of claim 18, wherein said second ingredient comprises at least one of a fatty acid and phenylalanine, and wherein said method further comprises ingesting said first and second ingredients at a predetermined period of time prior to eating a meal.

20. The method of claim 18, wherein said first and second ingredients are provided as an integral combination and wherein said method further comprises ingesting said first and second integral ingredients with a single swallow.
